(19) United States
(12) Patent Application Publication
(10) Pub. No.: US 2004/0106901 A1
(43) Pub. Date: Jun. 3, 2004

(54) Catheter having a balloon member invertedly attached thereto

(76) Inventors: William W. Letson, Alpharetta, GA (US); Donald J. McMichael, South Jordan, UT (US); Mark Foster, Sandy, UT (US)

Correspondence Address: KIMBERLY-CLARK WORLDWIDE, INC., 401 NORTH LAKE STREET, NEENAH, WI, 54956

(21) Appl. No.: 10/307,057

(22) Filed: Nov. 30, 2002

(51) Int. Cl. A61M 29/00

(52) U.S. Cl. 604/104; 604/96.01; 604/910; 604/271

(57) ABSTRACT

The present invention relates to balloon catheters wherein at least the proximal end of the balloon thereof is invertedly attached to the catheter shaft. Other aspects of the balloon catheters will have both ends of the balloon inverted. The present invention contemplates numerous other variations and modifications. As such, other aspects of the present invention will be apparent upon reading the remainder of the disclosure.

### BACKGROUND

Catheterization of a body cavity is frequently performed in medical procedures either to insert substances into or to remove substances from the body. During many of these procedures, it is necessary to keep the catheter in a relatively stable position to perform the desired insertion or removal. With the use of enteral feeding catheters (i.e., catheters which enable the administration of nutritional solutions directly into the stomach or intestines), for example, it is necessary to ensure that the catheter is not accidentally removed from the stomach or intestines. This is true both during the actual administration or removal of fluids, and the time periods in between.

In order to ensure that a catheter is maintained in the proper position, it is common to use a balloon disposed near the distal (patient) end of the catheter shaft. Inflating the balloon causes the balloon to contact the anatomical structure (i.e., a duct or stomach wall) and thereby prevents the catheter from moving out of the proper position. In the case of enteral feeding, a stoma is formed leading into the stomach or intestine. The catheter is positioned to extend through the stoma so as to form a channel into the stomach or intestines through which enteral feeding solutions may be instilled.

FIG. 1 shows a side view of a prior art balloon catheter 10 having a head 14 disposed at a proximal end 15 of the catheter 10. The head 14 contains valves (not shown) which regulate the flow of fluids through the balloon catheter 10. The head 14 also prevents the balloon catheter 10 from completely advancing through the stoma and into the stomach or intestine of the user.

To prevent the catheter 10 from being pulled out of the stomach/intestinal wall, a balloon 18 is disposed along a catheter shaft 26. The catheter 10 is shown having an optional stiff tip 30, which is attached to the catheter shaft 26 at a distal end 17 thereof opposite the head 14. The catheter shaft 26 is typically made of a medical grade silicone. The stiff tip 30, when present, is also frequently formed of a medical grade silicone but is usually configured to be as rigid as or less rigid than the catheter shaft 26.

The balloon 18 is advantageous because it allows the catheter shaft 26 to be inserted into the stoma (not shown) while the balloon 18 is uninflated. Once the catheter shaft 26 is properly positioned in the stoma, a syringe (not shown) is inserted into a side port 36 of the head 14 and a fluid is injected into the balloon 18 through a lumen (not shown in FIG. 1) of the catheter 10. The fluid inflates the balloon 18 so that it extends outwardly from the catheter shaft 26 and the stiff tip 30.

While the balloon 18 remains inflated, the catheter 10 stays properly positioned in the stoma. If the catheter 10 needs to be removed, the balloon 18 may be deflated so that it will not interfere with withdrawal of the catheter shaft 26 and stiff tip 30. The position of the balloon catheter 10 is maintained in such a manner until removal is desired. The type of balloon 18 shown in FIG. 1 is fashioned around the perimeter of the catheter shaft 26 such that when it is deflated it reduces or contracts about the shaft 26 but is still clearly larger than overall diameter of the catheter.

Attachment of the balloon 18 to the catheter shaft 26 is frequently accomplished by gluing the balloon proximal end 20 and the balloon distal end 22 to corresponding positions on the external surface of the catheter shaft 26 so as to form a proximal cuff 32 and a distal cuff 34, respectively. Such cuffs 32 and 34 are longitudinal sections of the balloon 18 whose inside diameters correspond to the outside diameter of the shaft 26 at their respective points of attachment to the catheter 10 and have a distance between them which is about the length of the uninflated balloon 18. The cuffs 32 and 34 must be of sufficient length to provide a tight and durable seal between the balloon 18 and the catheter shaft 26.

While the prior art balloon configuration shown in FIG. 1 works to maintain the balloon catheter 10 in the proper position within the patient, balloon catheters of this type as well as the other known balloon catheters do have disadvantages. For example, one drawback of prior balloon catheters is the attachment of the balloon 18 to the catheter 10 and the corresponding sizing issues. With regard to the catheter 10 of FIG. 1, the balloon 18 is attached to the catheter shaft 26, via proximal and distal cuffs 32 and 34, respectively, which extend out or away from the rest of the balloon 18 and which effectively increases the diameter of the shaft 26 at those points.

In use it is desirable to create as small a stoma as possible which will accommodate the catheter of choice yet will provide for a seal of the balloon 18 against the patient so as to avoid leakage. The existence of proximal extending cuff 32 adds to the diameter of the shaft 26 such that a larger stoma opening (not shown) will be required. That is in order to obtain the necessary contact or seal between the balloon 18 and the patient, the proximal cuff 32 will need to be positioned such that at least a portion of the proximal cuff 32 is within the stoma (not shown). Thus, a larger stoma opening is required than if the proximal cuff 32 or a portion thereof did not extend into the stoma when the catheter 10 was properly positioned within the patient.

Another disadvantage of prior balloon catheters is discomfort to the user. For example, with regard to the catheter of FIG. 1, in order to allow insertion of the catheter 10, the catheter shaft 26 and the stiff tip 30 must be relatively rigid or firm to prevent buckling under insertion pressures. However, this same firmness makes the distal tip 30 much more prone to irritate anatomical structures which come into contact with it. This is especially true in the stomach and intestines where the opposing walls of the anatomical structures tend to collapse on each other during physical exertion, or when the cavity has little or no food. As the person moves, the stiff tip 30 repeatedly engages the adjacent anatomical structure (such as the stomach wall) and can lead to irritation and/or discomfort for the user. Thus, as the presence of an extended stiff catheter tip in this environment has been suspected of irritating the opposing surfaces of the body cavity, it would be desirable if the patient could be protected from exposure to the tip 30.

Accordingly, there is a need in the art for a balloon catheter with a stiff distal tip isolated from opposing internal body cavity surfaces.

An additional disadvantage of prior catheters such as those in FIG. 1 is that the edges 35 and 37 of the cuffs 32 and 34, respectively present another edge which can catch on tissue as it passes into a patient or which can otherwise cause or lead to irritation.

Accordingly, there is a need for a catheter which allows for the creation of a sufficient seal between the catheter and the patient without the need for an increase in stoma size. There is also a need in the art for a catheter which will reduce the amount of patient contact with or exposure to rough or sharp edges which may be associated with the attachment of a balloon or sleeve to the catheter, thereby reducing or eliminating patient irritation caused thereby.

### SUMMARY OF THE INVENTION

In response to the difficulties and problems discussed above, a catheter having a means for expansion invertedly attached to the catheter shaft has been developed. More specifically, one aspect of this invention is directed to a catheter having a means for expansion having a proximal end and a distal end; and an elongate shaft having a distal end, a first lumen adapted for fluid communication, and a second lumen adapted for fluid communication with the means for expansion; wherein each end of the means for expansion is invertedly attached to the catheter.

Another embodiment of the present invention is directed to a balloon catheter having a head with at least two openings through which fluid may be passed; a shaft extending from the head, the shaft having a first and second lumen disposed in communication with the at least two openings, the catheter segment further having an interior and an exterior; and a sleeve having a proximal end and a distal end. At least the proximal end of the sleeve is attached to the shaft in an inverted or folded under fashion so as to form or create an expandable cavity between the sleeve and the catheter segment.

The present invention is also directed to a catheter including a head; an elongate shaft having a first lumen extending longitudinally therethrough, and a distal end; and an expandable sleeve having a first end and a second end. The ends of the sleeve are attached to the shaft in a folded under manner such that at least a portion of the attachments are significantly hidden when the sleeve is expanded.

Yet another aspect of the present invention is directed to a balloon catheter configured for placement through a stoma into a body cavity such that the balloon catheter may be maintained in the stoma. The balloon catheter may generally include a head having at least one opening through which a fluid may be introduced; a catheter shaft extending from the head, the catheter shaft having a distal end, an exterior and a wall defining a passageway through the interior; and a sleeve having a first end and a second end, where the ends are invertedly attached to the catheter shaft about the passageway so as to form an expandable balloon, such that the ends of the sleeve are substantially encompassed by the balloon when the balloon is expanded.

### BRIEF DESCRIPTION OF THE DRAWINGS

The above and other objects, features and advantages of the invention will become apparent from a consideration of the subsequent detailed description presented in connection with the accompanying drawings in which:

FIG. 1 shows a side view of a prior art balloon catheter, the balloon being in an inflated configuration.

FIG. 2 is a side view of one embodiment of the present invention, the catheter having the proximal end of the balloon or elongate sleeve attached to the catheter in an inverted fashion. The catheter is shown with an uninflated balloon.

FIG. 3 is a cross-sectional view of the catheter of FIG. 2.

FIG. 3A is an enlarged view of the portion of the catheter of FIG. 3 within the hashed circle;

FIG. 4 is a cross-sectional view of a means for expansion having annular rings therein;

FIG. 5 is a cross-sectional view of a catheter of the present invention having both ends of a means for expansion attached to the catheter shaft in an inverted manner;

FIG. 5A is an enlarged view of the portion of the catheter of FIG. 5 within the hashed circle;

FIG. 6 is a cross-section of a catheter of the present invention having a tip at the distal end of the catheter to which a means for expansion is attached;

FIG. 6A is an enlarged view of the portion of the catheter of FIG. 6 within the hashed circle;

FIG. 7 is a cross-sectional view of a catheter of the present invention having the distal end of a means for expansion attached at the distal end of the catheter; and

FIG. 8 is a cross-sectional view of a catheter of the present invention having a unitary component attached to the distal end of the catheter.

### DETAILED DESCRIPTION OF THE PRESENT INVENTION

Reference will now be made to the drawings in which the various elements of the present invention will be given numeral designations and in which the invention will be discussed so as to enable one skilled in the art to make and use the invention. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

As used herein, the term “distal” refers to the direction of the patient and the term “proximal” refers to the direction of the clinician.

It will be appreciated that while reference is made to a means for expansion, the term means for expansion is intended to mean or include, but is not limited to, a balloon, a sleeve, an inflatable or expandable member, an elastomeric sleeve, an expandable region or portion, an inflatable member, any other suitable expandable means, or the like. However, for ease of reading and understanding of this disclosure and not intending to be limited thereby, means for expansion will hereinafter be referred to as a balloon. It will also be appreciated that throughout the disclosure reference is made to inflation of the balloon, however, the present invention is not intended to be limited only to inflation. That is, while inflation is used herein for purposes of ease of reading and understanding the disclosure, the term inflation is also intended to mean or include, but is not limited to, expansion, enlargement, swelling or the like.

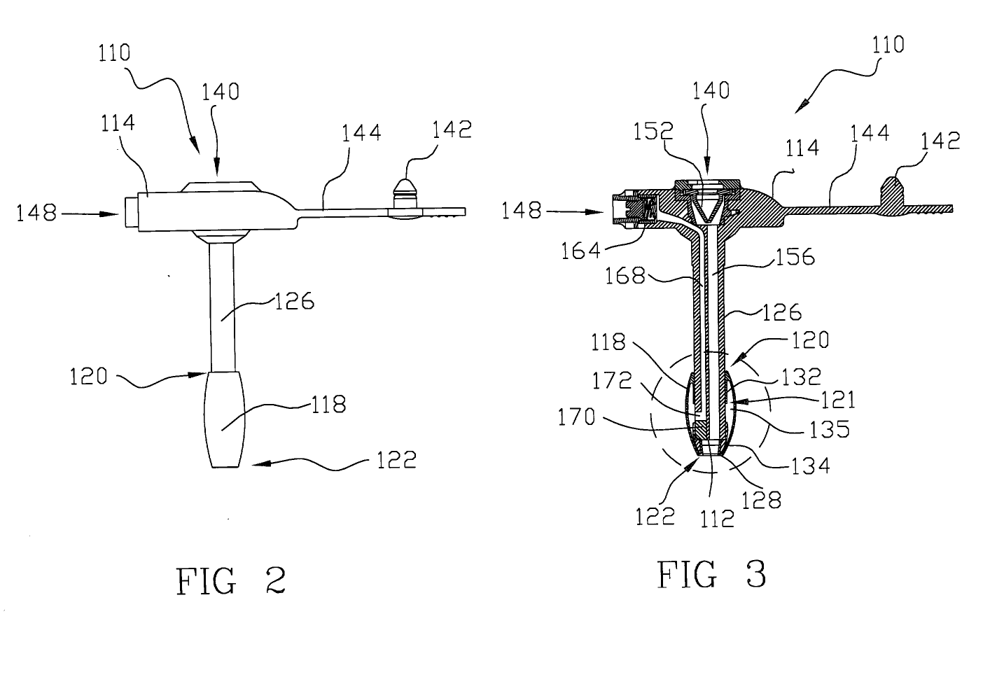

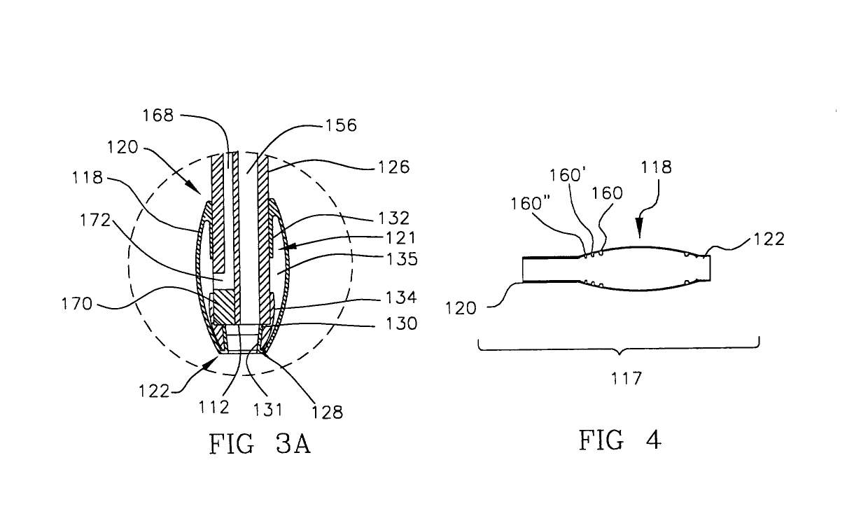

Referring now to FIGS. 2, 3, and 3A, there is shown a side view, a sectional view, and a partial sectional view, respectively, of a catheter 110 made in accordance with the teachings of the present invention. The catheter 110 generally includes a head 114 (FIGS. 2 and 3), a balloon 118, and a catheter shaft 126. The head 114 (FIGS. 2 and 3) has a proximal opening 140 (FIGS. 2 and 3) to a feeding lumen 156 (FIG. 3) within the shaft 126, for bolus feeding or providing other nutrient fluids, formula, and the like to a patient (not depicted). The catheter 110 (FIGS. 3 and 3A) also shows an optional stiff tip 130 (FIGS. 3 and 3A) attached at the distal end 112 of the catheter shaft 126. The stiff tip 130 (FIGS. 3 and 3A) has an interior surface 131 (FIG. 3A) which defines a passageway 160 which is configured for the passage of fluids, solutions, certain solids, or the like therethrough and into or out of the catheter 110 (FIGS. 2 and 3). An anti-reflux valve 152 (FIG. 3), which is generally included to prevent back-flow of the nutrient formula, is shown disposed between the opening 140 (FIGS. 2 and 3) and the inflation lumen 156 (FIG. 3). Inflation port 148 (FIGS. 2 and 3) is disposed in the head 114 (FIGS. 2 and 3) and communicates with inflation lumen 168 (FIG. 3) which extends longitudinally through the shaft 126 (FIGS. 2 and 3). The inflation lumen 156 (FIG. 3) is shown terminating laterally to the shaft 126 at port 172 (FIGS. 3 and 3A) into the cavity 135 (FIGS. 3 and 3A) created by the balloon 118 and the shaft 126.

A one-way valve 164 (FIG. 3) may be disposed between the inflation port 148 and inflation lumen 168 (FIG. 3). Application of positive fluid pressure, such as with air or saline, within and/or upon the inflation lumen 168 (FIG. 3) by way of the inflation port 148 (FIGS. 2 and 3) may cause the balloon 118 to inflate as the fluid fills the cavity 135 (FIGS. 3 and 3A) between the balloon 118 and the catheter shaft 126. Valve 164 (FIG. 3) helps prevent inadvertent deflation of the balloon 118. Also shown associated with the head 114 (FIGS. 2 and 3) is a plug 142 (FIGS. 2 and 3) for the proximal opening 140 (FIGS. 2 and 3) and a lanyard 144 (FIGS. 2 and 3) for retaining the plug 142 in a ready position. The plug 142 can be inserted in the opening 140 thereby reducing or precluding contamination when the opening 140 is not in use. Feeding lumen 156 (FIGS. 3 and 3A) extends longitudinally through shaft 126 and is shown terminating at the distal end 112 (FIGS. 3 and 3A) of the shaft 126.

It will be appreciated that the size and shape of the cavity 135 (FIGS. 3 and 3A) defined by or between the exterior of the shaft 116 and the balloon 118 may be varied during production or controlled by the user or clinician during use. Additionally, as discussed in more detail below the balloon 118 of the catheter 110 (FIGS. 2 and 3) may be designed to have a certain size and/or shape in either or both of its inflated or uninflated configurations. It will be understood and appreciated that varying the length of the balloon 118 and/or the points along the shaft 126 at which the ends 120 and 122 of the balloon 118 are attached may affect the shape of the resulting balloon.

Another suitable way of controlling the shape of the resulting inflated balloon includes, but is not limited to, annular rings, such as those shown at 160, 160′, and 160″ in FIG. 4, which may limit or promote expansion of the balloon in one or more directions. Other suitable ways of controlling the shape of the resulting balloon include, but are not limited to, those discussed in the U.S. Pat. No. 6,264,631 B1 to Willis et al., which is incorporated by reference in its entirety. For example, a plurality of annular rings may be disposed about the proximal end of the balloon. These rings force a greater volume of inflation medium into the distal end of the balloon, thus urging it to distend in the direction of the tip. In another embodiment, a plurality of centrally located annular rings bias radially against inflation of the balloon. These forces cause the balloon to distend longitudinally. The proximal distention is limited by the proximal body cavity wall. Thus, the distention over the distal tip is even more pronounced. In yet another embodiment, the uninflated balloon is longer than the portion of the shaft to which it is attached. This creates a longitudinal excess that allows the balloon to overlap the balloon cuffs and distend longitudinally upon inflation. As in the previously described embodiment, the proximal wall of the balloon enhances distal distention of the balloon. In still yet another exemplary embodiment, the balloon may have a thicker portion or wall at the proximal end and a thinner portion or wall at the distal end. The natural bias of the balloon adjacent the proximal cuff urges the bulk of the inflation to occur adjacent the distal cuff where the natural bias is relatively weak.

The various components of balloon catheter 110 may be made of any suitable material and may desirably be formed from bio-compatible materials such as medical grade silicone or the like. While valves 152 and 164 may be formed of any suitable material they are desirably made of a suitable polymer such as polycarbonate.

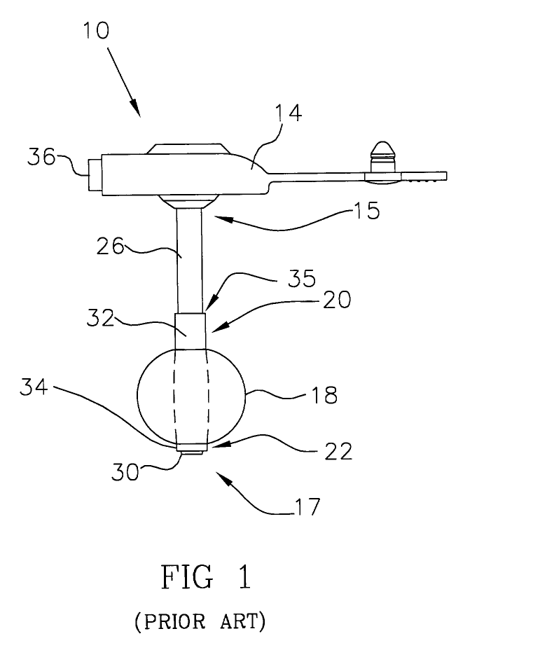

Unlike the prior art balloon catheter 10 shown in FIG. 1 which includes an attachment (such as proximal cuff 32) which extends in a generally longitudinal fashion along the catheter shaft 26 away from the portion of the balloon which expands, so as to be coaxial therewith, the proximal end 120 (FIGS. 3 and 3A) of the balloon 118 of the embodiment of the present invention shown in FIGS. 3 and 3A is invertedly attached to the catheter 110. While the proximal end 120 of the balloon 118 may extend, at least in part, longitudinally along the catheter shaft 126 so as to be coaxial therewith, and so as to form, for example, a proximal cuff 132, the proximal end 120 of the balloon 118 should be positioned along the shaft 126 relative to the balloon 118 such that at least part of the proximal end 120 of the balloon 118 is at least partially inverted or rolled under a portion of the remainder of the balloon 118. That is, as shown in FIGS. 3 and 3A, the side of the balloon 118 which is exposed to the user is the same side of the balloon 118 which is attached to the catheter shaft 126 at the proximal 120 end of the balloon 118.

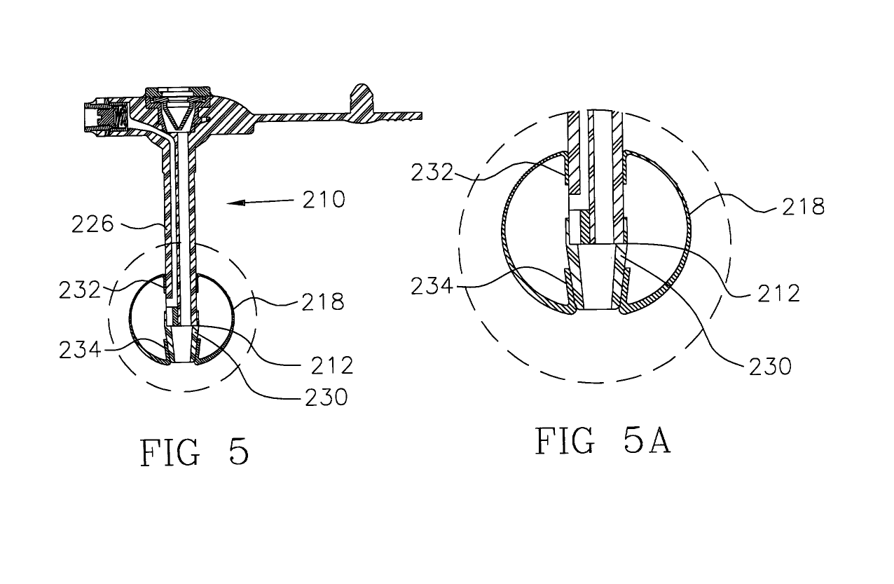

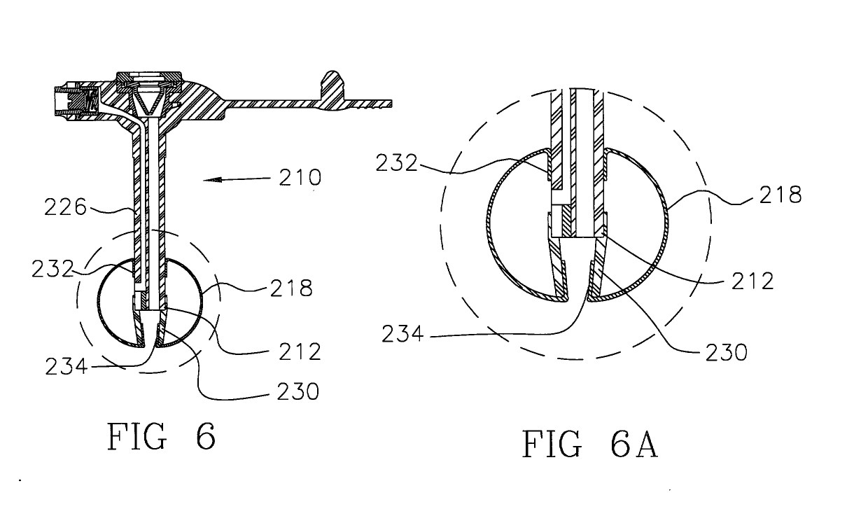

The distal end 122 (FIGS. 3 and 3A) of the balloon 118 may be secured to the catheter 110 in a fashion similar to that used to attach the proximal end 120 of the balloon 118. Thus, the attachment of the distal end 122 of the balloon 118 to the catheter shaft 126 may result in an inverted distal cuff 134. It will be appreciated that a balloon 118 may be attached to a catheter 110 in a variety of manners as well as a variety of locations. For example, the attachment of a balloon 118 may be achieved by forming a cuff 132 about a catheter 110 as shown in FIGS. 3 and 3A. It will be appreciated there are a number of different ways a cuff may be attached or secured to a catheter shaft 126, including but not limited to, those cuffs attached to the exterior of the catheter 110 as shown at 132 in FIGS. 3 and 3A and at 232 in FIGS. 5, 5A, 6 and 6A, and those cuffs 234 attached to the interior of the catheter 210 as shown in FIGS. 6 and 6A.

It will be appreciated that the size of the catheter 110 (FIGS. 2-4) as well as the length (inflated and uninflated) of the balloon 118 may be varied in accordance with the size and shape of the body cavity (not shown) the catheter 110 is to be used in and the nature of the matter to be moved through the catheter 110. That is, in some instances, it may be desirable to use catheters having larger and/or wider shafts than in other embodiments. Additionally, the balloon 118 of the catheter 110 may be designed to have a certain size and/or shape in either or both of its inflated or uninflated configurations.

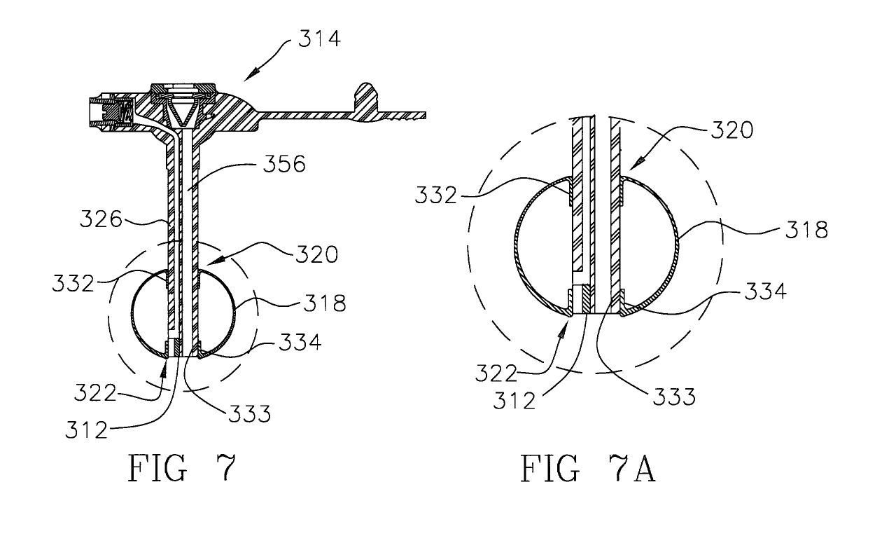

Additionally, as indicated above, the present invention also contemplates the inclusion of a tip 130 (FIGS. 3 and 3A) which may be attached to the distal end 112 (FIGS. 3 and 3A) of the shaft 126. It is contemplated that the tip 130 may be part of the shaft (e.g., formed integrally with the shaft) or may be a separate attachment (FIGS. 2, 3 and 3A). Where a tip 130 is present the distal end 122 of the balloon 118 may be attached to the tip 130 or to the shaft 126. When present either as an integral part of the catheter shaft 126 or separately formed and later attached, the tip 130 should be considered to be a part of the catheter 110. That is, for example, each embodiment should be considered to be attached to the catheter whether attached directly to the catheter or the tip. As further clarification, if, for example, at least one of the ends of a balloon is said to be attached to the exterior of a catheter, such that the distal end 222 (FIGS. 5 and 5A) of the balloon 218 (FIGS. 5 and 5A) is attached to the exterior of the tip 230 (FIGS. 5 and 5A) or to the exterior of the catheter shaft 326 (FIG. 7), each embodiment should be considered to be attached to the catheter whether attached directly to the catheter or the tip. The same is contemplated for interior attachment to the shaft or tip of a catheter.

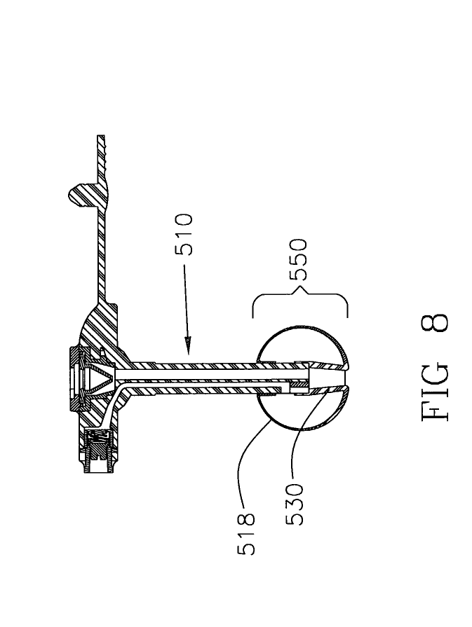

While there has been discussion above concerning attachment of a balloon to the interior or to the exterior of catheter shaft, the distal end of the balloon may be attached (not shown) to the distal end of the tip. Further still, rather than necessitating that the tip and the balloon be created separately from one another, it is also contemplated that the tip 530 may be part of a unitary component 550 (FIG. 8), where the unitary component 550 includes a tip 530 integrally formed with a balloon 518. A more detailed description and discussion of exemplary unitary components may be found in commonly owned and co-pending U.S. patent application Ser. No. __/______ (Attorney Docket No.17,110A), Ser. No. __/______ (Attorney Docket No.17,110B), and Ser. No. __/______ (Attorney Docket No.17,110C), entitled “CATHETER WITH UNITARY COMPONENT”, “PROCESS FOR SECURING A TIP MEMBER TO CATHETER DURING PRODUCTION OF THE TIP MEMBER” and “PROCESS FOR PRODUCING UNITARY COMPONENT AND A CATHETER HAVING A UNITARY COMPONENT”, respectively, each to McMichael et al., and each filed Nov. 30, 2002, all of which are incorporated by reference in their entirety herein.

As will be appreciated, the catheters described above as well as those contemplated to be within the scope of the disclosure and claims of the present invention have several advantages over those prior catheters. For example, as a result of the inversion of at least the proximal end 122 (FIGS. 3 and 3A) of the balloon 118, it will be possible for users of catheters of the present invention to utilize smaller stoma openings (not shown) as the proximal end 120 and cuff 132 of the balloon 118 will no longer extend away from the remainder of the balloon 118 as with previous devices. As such the user will be able to create the necessary seal between the patient (not shown) and the catheter 110 (FIGS. 2, 3 and 3A) without having all or part of a cuff 132 (FIGS. 3 and 3A) of the balloon 118 (FIGS. 2, 3 and 3A) being in the stoma (not shown) when the catheter 110 is properly positioned. Additionally, by avoiding a balloon cuff 132 in the stoma of the patient, the balloon 118 of the present invention may be able to provide a better seal for at least two reasons. First, the stoma (not shown) may be smaller and thus fluids (if present at the stoma site) now have a smaller opening to pass through. Second, the inverted proximal end 122 (FIGS. 3 and 3A) of the balloon 118 may provide for or allow the creation of a balloon shape which was not previously obtained with prior balloon catheters but which also enables the creation of a better seal with the patient and/or which requires less pressure to be exerted on the patient by the balloon.

Further still, while the ends of a balloon may be inverted on catheter shafts having a variety of lengths, a balloon having one or more ends invertedly attached to a catheter shaft is suited for use with shorter catheter shafts than were previously possible with the prior art balloon catheters. While the shaft of a catheter of the present invention will need to be of a length sufficient to accommodate the balloon and enable the catheter's purpose the catheter need be no longer. That is, the inversion of one or more of the ends of the balloon may reduce the length of shaft which is necessary, as compared with prior devices, as the shaft of the present invention need not have additional length added to the shaft so as to accommodate balloon cuffs which extend away from the remainder of the balloon at both ends. As suggested above, this reduction of catheter shaft length may decrease or minimize irritation associated with prior art devices. That is, as the catheter shaft or segment is shorter, the distal end or tip (depending on the embodiment) of the catheter may be less likely to come in contact with the opposite side of the body cavity into which the catheter is placed, and therefore less likely to cause irritation and/or discomfort associated with such contact. As such the want or need for a distally extending or protruding balloon or sleeve may, in some instances, be eliminated, although the use of a distally extending or protruding balloon is still generally desirable.

It will be appreciated that it is not intended that the use of a balloon which has one or more of its ends invertedly attached to a catheter shaft can and should only be used with catheters which are sized no larger than necessary to accommodate the balloon and/or its ends, rather the present invention provides the ability to use shorter catheter shafts should that be of interest to the clinician.

Further still, the use of a catheter having one or more inverted ends can result in less irritation upon insertion and/or removal of the device as well as potentially reducing infection. That is, for example, at each end 120 or 122 (FIGS. 3 and 3A) of a balloon 118, there is an edge 121 or 123, respectively. Thus, as at least one end 120 or 122 (FIGS. 3 and 3A) of a balloon 118 of the present invention is attached to the catheter shaft 126 in an inverted or folded under fashion, at least one edge 121 or 123 of the balloon 118 will not be exposed to the patient. This is of note as the prior art catheters typically had two exposed edges which could catch on tissue as it passed into or out of a patient or which could otherwise cause or lead to irritation. As a result of the ends 120 or 122 (FIGS. 3 and 3A) of the balloon 118 being inverted, reversed or folded under as shown and discussed above, the catheters of the present invention will reduce, if not eliminate, a patient's contact with or exposure to potentially rough edges at the end 120 or 122 of a balloon 118. This reduction in contact or exposure may lead to a reduction or elimination of patient irritation caused by previous attachments or cuffs 32 (FIG. 1) or their associated edges 35 (FIG. 1). The reduction or elimination of exposed edges will also reduce the available surface area for bacteria and the like to accumulate and thus the potential for infection may also be reduced with the catheters of the present invention.

As noted briefly above another embodiment of the present invention is directed to a balloon catheter having a head having at least two openings through which, for example, a fluid or nutritional solution may be passed; a catheter shaft extending from the head, the shaft having at least a first and second lumen, each lumen being disposed in communication with one of the at least two openings, the shaft further having an interior and an exterior; and a sleeve having a proximal end and a distal end. At least the proximal end of the sleeve should be folded under or reversely attached to the catheter shaft so as to form or create an expandable cavity between the sleeve and the catheter shaft. Some embodiments of this aspect of the invention may also include a tip attached to the distal end of the catheter shaft.

An additional embodiment of the present invention noted above relates to a catheter including a head, an elongate shaft, and an expandable sleeve. The elongate shaft has a first lumen extending longitudinally therethrough, and a distal end. The expandable sleeve has a distal end and a proximal end. The ends of the sleeve are attached to the shaft in a folded under manner such that the ends of the sleeve are at least significantly hidden when the sleeve is expanded. FIGS. 5 and 5A illustrates how, in one embodiment, the ends of a sleeve may be significantly hidden when the sleeve is expanded.

One or more embodiments of the present invention may be produced such that the sleeve is located between the head and the distal end of the catheter. In such a case, the sleeve may be such that when expanded the entire sleeve remains between the head and distal end of the catheter. Alternately, the sleeve may be configured such that when expanded it extends in part beyond the distal end of the catheter or tip (if present). In those embodiments which do not have one end of the balloon or sleeve attached to the interior of the catheter, it is desirable for the balloon or sleeve to have at least one cuff or point of attachment that is located at or near the distal end of the catheter.

Yet another aspect of the present invention is directed to a balloon catheter configured for placement through a stoma (not shown) into a body cavity (not shown) so that the balloon catheter may be maintained in the stoma. As shown in FIG. 7, the balloon catheter 310 includes a head 314, a catheter shaft 326; and a sleeve. The head has at least one opening 340 through which a fluid may be introduced. The catheter shaft 326 has a distal end 312, an exterior, and an interior surface 333 which defines a passageway 356 through the shaft 326. The shaft 326 extends from the head 314 of the catheter 310. The sleeve is invertedly attached to the catheter shaft 326 about the passageway so as to form an inflatable balloon 318, such that the inverted attachments 332 and 334 are substantially encompassed by the remainder of the balloon 318 when the balloon 318 is expanded.

As suggested above, there are many ways for the sleeve to be attached to the catheter shaft 326, including, for example, where the first end 320 of the sleeve is attached to the catheter shaft 326 so as to form a proximal cuff 332 of the balloon 318, and where the second end 322 of the sleeve is attached to the catheter shaft 326 so as to form a distal cuff 334 of the balloon 318. Although not shown in the figures, other suitable attachments of the sleeve to the catheter shaft 326 include, but are not limited to, those where the distal end 322 of the sleeve is attached to the distal end 312 of the catheter 310 such that the distal end 322 of the sleeve defines a portion of the passageway through the catheter shaft.

Further embodiments of the invention may include a catheter shaft 326 having a tip (not shown) attached to the distal end 312 of the catheter shaft 326, opposite the head 314. As noted above, in such embodiments, one possible way of orienting the attachment of the sleeve 318 is such that the proximal end 320 of the sleeve 318 is attached to the exterior of the catheter shaft 326 and the distal end 322 of the sleeve 322 is attached interior of the stiff tip (not shown).

It will be appreciated that each embodiment of the present invention may not possess each and every component described or contemplated hereby and/or may not possess each and every advantage described or contemplated herein but all such embodiments are nevertheless contemplated to be within the scope of the disclosure and the attached claims.

While various patents and other reference materials have been incorporated herein by reference, to the extent there is any inconsistency between incorporated material and that of the written specification, the written specification shall control. In addition, while the invention has been described in detail with respect to specific embodiments thereof, those skilled in the art, upon obtaining an understanding of the invention, may readily conceive of alterations to, variations of, and equivalents to the described embodiments. It is intended that the present invention include such modifications and variations as come within the scope of the appended claims and their equivalents.

We claim:

1. A catheter comprising:
a means for expansion having a proximal end and a distal end; and
an elongate shaft having a distal end, a first lumen adapted for fluid communication and a second lumen adapted for fluid communication with the means for expansion;
wherein each end of the means for expansion is invertedly attached to the catheter.

2. The catheter of claim 1, wherein at least one of the inverted ends of the means for expansion is a cuff.

3. The catheter of claim 1, wherein expansion of the means for expansion occludes at least a portion of one of the invertedly attached ends.

4. The catheter of claim 1, wherein the distal end of the shaft further comprises a tip.

5. The catheter of claim 1 further comprising a unitary component having a tip integrally formed with the means for expansion, wherein the tip is attached to the distal end of the shaft.

6. The catheter of claim 4, wherein the tip may be part of the shaft or a separate attachment.

7. The catheter of claim 4, wherein the distal end of the means for expansion is attached to the tip.

8. The catheter of claim 1, the shaft further comprising an interior and an exterior, wherein at least one of the invertedly attached ends of the means for expansion is attached to the interior of the catheter.

9. The catheter of claim 1, wherein the means for expansion is a balloon, a sleeve, or an expandable member.

10. A balloon catheter comprising:
a head having at least two openings through which fluid may be passed;
a shaft extending from the head, the shaft having a first and second lumen disposed in communication with the at least two openings, the shaft further having an interior and an exterior; and
a sleeve having a proximal end and a distal end, wherein at least the proximal end of the sleeve is attached to the shaft in a folded under fashion;
wherein an expandable cavity is formed between the sleeve and the shaft.

11. The catheter of claim 9, wherein the proximal end of the sleeve is attached to the exterior of the shaft, and the distal end of the sleeve is attached to the interior of the shaft.

12. The catheter of claim 9, wherein the proximal end of the sleeve is attached to the exterior of the shaft, and the distal end of the sleeve is attached to the exterior of the shaft.

13. The catheter of claim 9, wherein
the shaft has a distal end; and
wherein the catheter further comprises a tip attached to the distal end of the catheter shaft.

14. The catheter of claim 13, wherein the proximal end of the sleeve is attached to the exterior of the catheter shaft, and wherein the distal end of the sleeve is attached to the tip.

15. A catheter comprising:
a head;
an elongate shaft having at least a first lumen extending longitudinally therethrough, and a distal end; and
an expandable sleeve having a first end and a second end;
wherein the ends of the sleeve are attached to the shaft in a folded under manner such that the ends of the sleeve are at least significantly hidden when the sleeve is expanded.

16. The catheter of claim 15, wherein the sleeve is located between the head and the distal end of the catheter.

17. The catheter of claim 15, wherein the sleeve has at least one cuff, and wherein one of the cuffs is located at or near the distal end of the catheter.

18. The catheter of claim 15, wherein the sleeve has at least one cuff, and wherein at least one of the cuffs is attached to the interior of the catheter.

19. A balloon catheter configured for placement through a stoma into a body cavity, the balloon catheter comprising:
a head having at least one opening through which a fluid may be introduced;
a catheter shaft extending from the head, the catheter shaft having a distal end, an exterior and a wall defining a passageway through the interior; and
a sleeve having a first end and a second end, the ends being invertedly attached to the catheter shaft about the passageway so as to form an expandable balloon, such that the ends of the sleeve are substantially encompassed by the balloon when the balloon is expanded.

20. The balloon catheter of claim 19, wherein the first end of the sleeve is attached to the catheter shaft so as to form a proximal cuff, and wherein the second end of the sleeve is attached to the catheter shaft so as to form a distal cuff.

21. The balloon catheter of claim 19, wherein the distal end of the sleeve is attached to the distal end of the catheter, and wherein the distal end of the sleeve defines a portion of the passageway through the catheter shaft.

22. The balloon catheter of claim 19 further comprising a tip attached to a distal end of the catheter shaft.

23. The balloon catheter of claim 22, wherein the sleeve is attached to the interior of the tip and the exterior of the catheter shaft.
